(19) United States
(12) Patent Application Publication
(10) Pub. No.: US 2023/0218657 A1
(43) Pub. Date: Jul. 13, 2023

(54) COMPOSITIONS, THEIR USES IN THERAPY AND METHODS THEREOF

(71) Applicants: National University of Singapore, Singapore (SG); Singapore Health Services Pte Ltd, Singapore (SG)

(72) Inventors: Veronique Sandrine Angeli, Singapore (SG); Bien Keem Tan, Singapore (SG)

(21) Appl. No.: 18/000,492

(22) PCT Filed: Jun. 2, 2021
(86) PCT No.: PCT/SG2021/050318

§ 371 Date: Dec. 1, 2022

(30) Foreign Application Priority Data
Jun. 2, 2020 (SG) 10202005191X

(51) Int. Cl. A61K 31/724; A61P 7/10

(52) U.S. Cl. CPC A61K 31/724; A61P 7/10; A61K 9/0021

(57) ABSTRACT

The present invention relates to a method of treating lymphedema comprising administering an effective amount of a composition to a patient in need thereof. The composition consisting essentially of cyclodextrin or a pharmaceutically acceptable salt, solvate or prodrug thereof. The present invention also relates to a composition consisting essentially of cyclodextrin.

### TECHNICAL FIELD

The present invention relates, in general terms, to compositions and their uses in therapy. The present invention also relates to methods of treatment thereof.

### BACKGROUND

Lymphedema, also known as lymphoedema and lymphatic edema, is a condition of localized swelling caused by a compromised lymphatic system. The lymphatic system functions as a critical portion of the body’s immune system and returns interstitial fluid to the bloodstream. In general, the swelling occurs at the arms, hands, legs, breast or torso. Lymphedema is most frequently a complication of cancer treatment, parasitic infections, trauma, but it can also be observed in a number of genetic disorders. Tissues with lymphedema are at high risk of infection, fibrosis and malignant transformations because the lymphatic system has been compromised.

Lymphedema can be further classified as primary (genetic) lymphedema or secondary lymphedema (caused by cancer, infection etc). Primary lymphedema is a rare, inherited condition caused by problems with the development of lymph vessels in the body. Specific causes of primary lymphedema include Milroy’s disease (congenital lymphedema), Meige’s disease (lymphedema praecox) and late-onset lymphedema (lymphedema tarda).

Secondary lymphedema is a debilitating and chronic inflammatory disease caused by poor lymphatic drainage caused by cancer treatment (surgery and/or radiotherapy), injury, trauma or infection. Examples of such causes include surgery, radiation treatment for cancer, cancer and infection. Lymphedema can also result from a blockage in the lymphatic system. The blockage prevents lymph fluid from draining well, and the fluid build-up leads to swelling.

The swelling caused by lymphedema ranges from mild, hardly noticeable changes in the size of your arm or leg to extreme changes that make the limb hard to use. Lymphedema caused by cancer treatment may not occur until months or years after treatment.

There is presently no cure for lymphedema. In particular, there is currently no medication that is suitable for treating lymphedema. Physical, treatment such as compression therapy, good skin care, exercise, and manual lymphatic drainage are commonly recommended to improve the outcome.

It would be desirable to overcome or ameliorate at least one of the above-described problems.

### SUMMARY

The present invention is predicated on the understanding that patients with cancer-associated lymphedema also have symptoms such as inflammation and fibrosis which occur during the late stages of the disease. This results from dying adipocytes which are not able to store the excess of lipids that accumulates in the tissue as the consequence of impaired lymphatic clearance. Based on these findings, the inventors postulated that clearing lipids in a body of an organism will reverse lymphedema and associated tissue changes such as fibrosis and adipocyte loss. It was found that an intradermal injection of cyclodextrin in an animal model of lymphedema was effective in reducing lymphedema. In particular, treatment with cyclodextrin was able to significantly reduce lipid accumulation in tissue, thus reducing tissue swelling and restore lymphatic drainage.

The present invention provides a method of treating lymphedema comprising administering an effective amount of a composition to a patient in need thereof, the composition comprising cyclodextrin or a pharmaceutically acceptable salt, solvate or prodrug thereof;

wherein an interior cavity of the cyclodextrin is unoccupied.

The present invention also provides a composition for treating lymphedema in a patient in need thereof, the composition comprising cyclodextrin or a pharmaceutically acceptable salt, solvate or prodrug thereof;

wherein an interior cavity of the cyclodextrin is unoccupied.

The present invention also provides a use of a composition in the manufacture of a medicament for treating lymphedema, the composition comprising cyclodextrin or a pharmaceutically acceptable salt, solvate or prodrug thereof;

wherein an interior cavity of the cyclodextrin is unoccupied.

In some embodiments, the composition to be administered ameliorates or reduces tissue edema, lymphatic function, lipid accumulation, tissue cholesterol, fibrosis, inflammation, or a combination thereof.

In some embodiments, the composition is to be administered on alternate days.

In some embodiments, the composition is to be administered in at least 2 doses over at least 4 days.

In some embodiments, the composition is to be administered at a dose of about 0.5 g/kg to about 10 g/kg.

In some embodiments, the composition is to be administered to a patient in need thereof who has lymphedema with a severity of stage 2 or less.

The present invention also provides a composition, the composition comprising cyclodextrin;

wherein an interior cavity of the cyclodextrin is unoccupied.

In some embodiments, a weight ratio of cyclodextrin to the composition is about 0.1%w/w to about 50%w/w.

In some embodiments, the cyclodextrin is derivatised with alkyl, alkenyl, alkynyl, alkoxy, hydroalkyl, halo, haloalkyl, haloalkenyl, haloalkynyl, alkylacyl, alkenylacyl, alkynylacyl, aryl, or alkylaryl.

In some embodiments, the cyclodextrin is derivatised with methyl, ethyl, propyl, hydromethyl, hydroethyl, hydropropyl, fluoro, chloro, bromo, benzyl, or phenyl.

In some embodiments, the cyclodextrin is derivatised 1 to 24 times.

In some embodiments, the cyclodextrin is a β-cyclodextrin selected from methyl-β-cyclodextrin, 2-hydroxylpropyl-β-cyclodextrin, or a combination thereof.

In some embodiments, the interior cavity of the cyclodextrin has a diameter of about 5.5 Å to about 9.7 Å.

In some embodiments, the interior cavity of the cyclodextrin is unoccupied or occupied with an inert molecule.

In some embodiments, the composition further comprising an excipient selected from stabilisers, solubilisers, emulsifiers, surfactants, water-soluble polymers, pH modifiers, fillers, binders, pigments, disintegrants, antioxidants, preservatives, emollients, silicones, penetration enhancers, lubricants and perfume.

In some embodiments, the excipient is selected from microcrystalline cellulose; metallic salts of acids such as aluminium stearate, calcium stearate, magnesium stearate, sodium stearate, and zinc stearate; fatty acids, hydrocarbons and fatty alcohols such as stearic acid, palmitic acid, liquid paraffin, stearyl alcohol, and palmitol; fatty acid esters such as glyceryl (mono- and di-) stearates, triglycerides, glyceryl (palmitic stearic) ester, sorbitan monostearate, saccharose monostearate, saccharose monopalmitate, and sodium stearyl fumarate, alkyl sulfates such as sodium lauryl sulfate and magnesium lauryl sulfate; polymers such as polyethylene glycols, polyoxethylene glycols, and polytetrafluoroethylene, and inorganic materials such as talc and dicalcium phosphate, and sodium starch glycolate.

In some embodiments, the composition is an intradermal composition.

In some embodiments, the intradermal composition comprises an aqueous medium.

In some embodiments, the intradermal composition comprises a saline solution.

In some embodiments, the aqueous medium is selected from 0.9% NaCl saline solution 0.9% KCl saline solution, Ringer’s lactate solution, Acetated Ringer’s solution, Intravenous sugar solutions, 5% dextrose in normal saline (D5NS), 10% dextrose in normal saline (D10NS), 5% dextrose in half-normal saline (D5HNS), 10% dextrose in half-normal saline (D10HNS), Phosphate buffered saline (PBS), TRIS-buffered saline (TBS), Hank’s balanced salt solution (HBSS), Earle’s balanced salt solution (EBSS), Standard saline citrate (SSC), HEPES-buffered saline (HBS), and Gey’s balanced salt solution (GBSS).

In some embodiments, the weight ratio of the aqueous medium to the composition is about 0.1%w/w to about 50%w/w.

In some embodiments, the intradermal composition comprises an excipient selected from carboxymethylcellulose sodium, microcrystalline cellulose, cresol, methylparaben, and propylparaben.

In some embodiments, the weight ratio of excipients to intradermal composition is about 0.1%w/w to about 50%w/w.

In some embodiments, the composition is a topical composition.

In some embodiments, the topical composition is a topical cream or topical gel.

In some embodiments, the topical composition further comprises an excipient selected from a carrier, a emulsifier and/or a thickening agent.

In some embodiments, the carrier is selected from mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water.

In some embodiments, the topical composition further comprises an excipient is selected from Arabic gum, acetic acid, acetone, acetyltributyl citrate, agar, ethanol, alginic acid, almond oil, alpha tocopherol, aluminium monostearate, aluminium stearate, aluminium oxide, ascorbic acid, ascorbyl palmitate, bentonite, benzalkonium chloride, benzethonium chloride, benzoic acid, benzyl alcohol, benzyl benzoate, boric acid, bronopol, butylated hydroxyanisole, butylated hydroxytoluene, butylene glycol, butylparaben, calcium acetate, calcium alginate, calcium chloride, calcium hydroxide, calcium lactate, carbomer, carboxymethylcellulose sodium, carrageenan, castor oil, microcrystalline cellulose, ceresin, cetostearyl alcohol, cetrimide, cetyl alcohol, chlorocresol, chloroxylenol, chlorosterol, citric acid, colloidal silicon dioxide, cresol, crospovidone, cyclomethicone, denatonium benzoate, dibutyl phthalate, diethanolamine, dimethicone, dimethyl phthalate, dimethylacetamide, disodium edetate, docusate sodium, ethyl acetate, ethyl lactate, ethyl oleate, ethylene vinyl acetate, ethylparaben, gelatin, glycerine, glyceryl monooleate, glyceryl monostearate, glycofurol, hydroxyethyl cellulose, hydroxypropyl cellulose, hypromellose, imidurea, isopropyl alcohol, isopropyl myristate, isopropyl palmitate, kaolin, lactic acid, lanolin, lecithin, linoleic acid, silicate, malic acid, mannitol, methylcellulose, methylparaben, monothioglycerol, myristic acid, myristyl alcohol, oleic acid, oleyl alcohol, palmitic acid, paraffin, petrolatum, phenoxyethanol, poloxamer, polycarbophil, polyethylene glycol, polymethylacrylate, polyoxyglyceride, polyvinyl alcohol, povidone, propylene glycol, propylparaben, pyrrolidone, sodium hydroluronate, sodium lactate, sodium lauryl sulfate, sorbitan ester, sorbitol, starch, tricaprylin, triethylanolamine, xanthan gum, or xylitol.

In some embodiments, the weight ratio of excipients to topical composition is about 0.1%w/w to about 50%w/w.

In some embodiments, the composition is applied to a transdermal patch.

In some embodiments, the composition is applied as a layer within the transdermal patch or applied on a surface of the transdermal patch.

### BRIEF DESCRIPTION OF THE DRAWINGS

Embodiments of the present invention will now be described, by way of non-limiting example, with reference to the drawings in which:

FIG. 1 illustrates images of footpads from female wild-type (WT) or apoE-/-mice after receiving subcutaneous injection of cyclodextrin or saline;

FIG. 2 illustrates microscopic images of female WT or apoE-/- mice after receiving subcutaneous injection of cyclodextrin or saline;

FIG. 3 plots the relative fluorescence in the lymph nodes of respective female WT or apoE-/- mice after receiving subcutaneous injection of cyclodextrin or Saline;

FIG. 4 plots the relative fluorescence in the lymph nodes of the respective mice under a different dosage regime;

FIG. 5 shows the structure of cyclodextrins;

FIG. 6 shows the total cholesterol concentration analysed in the front footpad of wild-type, apoE-/- mice, and apoE-/- mice after treatment;

FIGS. 7 shows staining images of the back skin of treated and untreated mice;

FIG. 8A shows lymphatic function assessed in treated and untreated mice; and

FIG. 8B shows footpad tissue swelling of treated and untreated mice.

### DETAILED DESCRIPTION

The present invention is predicated on the understanding that if lipids can be cleared from a body of an organism, lymphedema can be reversed and associated tissue changes such as fibrosis and adipocyte loss. Without wanting to be bound by theory, the inventors believe that cyclodextrins, due to their frustum morphology, can solubilize lipids and extract them from cells. To this end, it was found that treatment of an organism with lymphedema like conditions with cyclodextrin was able to significantly reduce lipid accumulation in tissue, thus reducing tissue swelling and restore lymphatic drainage.

This is advantageous as the current treatment for lymphedema are limited to either garment or microsurgery. There is no drug therapy. In this regard, the composition can be formulated as a drug treatment for lymphedema.

Additionally, cyclodextrin will help to clear protein and lipid and therefore will reduce inflammation and fibrosis. Because of this, cyclodextrin is also expected to improve efficiency of current microsurgeries aiming at restoring lymphatic transport including lymphatic-vein anastomosis and lymph node transplant. Cyclodextrin is also non-toxic and thus can facilitate ethical and clinical trial approval.

Accordingly, the present invention provides a composition, the composition consisting essentially of cyclodextrin.

As used herein, “cyclodextrins” refer to a family of cyclic oligosaccharides, consisting of a macrocyclic ring of glucose subunits joined by α-1,4 glycosidic bonds. Cyclodextrins are composed of 5 or more α-D-glucopyranoside units linked 1->4, as in amylose (a fragment of starch). The largest cyclodextrin that was found contains 32 1,4-anhydroglucopyranoside units. More commonly, cyclodextrins contain a number of glucose monomers ranging from six to eight units in a ring, creating a cone shape. Cyclodextrins with 6 glucose subunits are also known as α (alpha)-cyclodextrin, 7 glucose subunits as β (beta)-cyclodextrin, and 8 glucose subunits as γ (gamma)-cyclodextrin. The cyclodextrins have toroidal shapes, with the larger and the smaller openings of the toroid exposing to the solvent secondary and primary hydroxyl groups respectively. Because of this arrangement, the interior of the toroids is not hydrophobic, but considerably less hydrophilic than the aqueous environment and thus able to host other hydrophobic molecules. In contrast, the exterior is sufficiently hydrophilic to impart cyclodextrins (or their complexes) water solubility. They are not soluble in typical organic solvents.

In some embodiments, the cyclodextrin is selected from α-cyclodextrin, β-cyclodextrin, γ-cyclodextrin or a combination thereof.

As used herein, cyclodextrins also include its derivatives thereof. Derivatization is a technique used in chemistry which converts a chemical compound into a product (the reaction’s derivate) of similar chemical structure, called a derivative. Generally, a specific functional group of the compound participates in the derivatization reaction and transforms the educt to a derivate of deviating reactivity, solubility, boiling point, melting point, aggregate state, or chemical composition. The hydroxyl groups on cyclodextrins can be chemically modified, and thus also modify its host-guest behaviour. For example, conversions such as O-methylation and acetylation can be used. Hydroxypropylated derivatives can also be formed using propylene oxide. The primary alcohols can also be tosylated.

In some embodiments, the cyclodextrin is optionally derivatised. The optional derivatives can be selected from alkyl, alkenyl, alkynyl, alkoxy, hydroalkyl, halo, haloalkyl, haloalkenyl, haloalkynyl, alkylacyl, alkenylacyl, alkynylacyl, aryl, or alkylaryl. The optional derivatives can be selected from methyl, ethyl, propyl, hydromethyl, hydroethyl, hydropropyl, fluoro, chloro, bromo, benzyl, or phenyl. Other types of derivatised cyclodextrins are shown in Table 1 below:

TABLE 1

a-cyclodextrin derivatives

| | | | | |
|---|---|---|---|---|
| α-Cyclodertrin Derivative | Short Name | Molecular Formula | Molecular Weight | DS |
| native | | C₃₆H₆₆O₃₀ | 972.84 | 0 |
| random methyl | RAMEA | C₄₇H₈₂O₃₀ | 1126.9 | -11 |
| hexahis(2,3-tri-O-methyl) | TRIMEA | C₅₄H₉₆O₃₀ | 1225.4 | 18 |
| (2-hydroxy)propyl | HPACD | C_{49.5}H₆₇O_{34.5} | 1234.3 | -4.5 |
| sulfated Na-salt | sulfated | C₃₆H₄₉O₆₆S₁₂Na₁₂ | 2197.4 | -12 |
| phosphated Na-salt | phosphated | C₃₆H₆₆O₄₂P₄Na₄ | 1350.7 | -2-6 |
| carboxymethylated Na-salt | CMACD | C₄₈H₆₃O₃₆Na₃ | 1212.9 | -3.5 |
| succinylated | SuACD | C₅₂H₇₆O₄₂ | 1373.2 | -4 |
| acetylated | AcACD | C₅₂H₇₆O₃₆ | 1267.1 | -7 |
| carboxymethyl-α-CD crosslinked with epichlorohydrin | CMACDEp | 56 kDa | | |

While Table 1 shows derivatives of α-cyclodextrins, the skilled person would understand that β-cyclodextrins and γ-cyclodextrins can also be similarly derivatised.

In other embodiments, the derivatised cyclodextrin is randomly substituted. Randomly substituted cyclodextrin derivatives are modified at various positions and they can be characterised by degree of substitution.

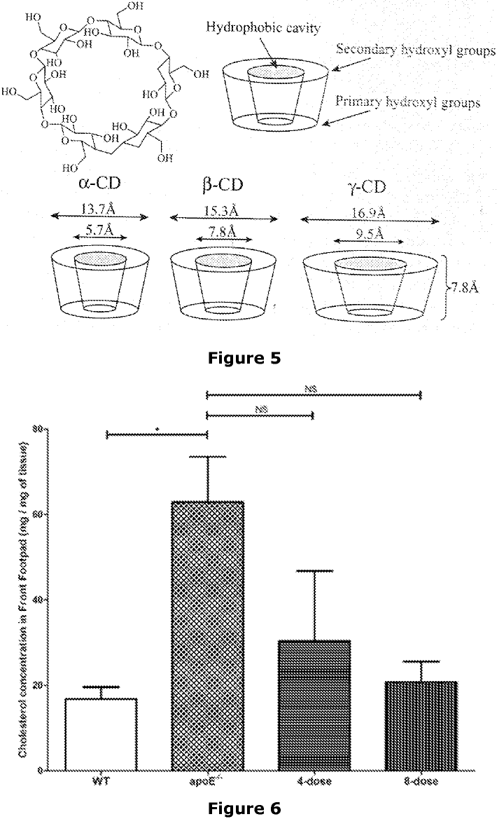

The degree of derivatization can be adjusted. For example, cyclodextrin can be fully methylated or partially methylated. In the case of α-cyclodextrin, 6 secondary hydroxyl groups are exposed relative to the frustum shaped compound (FIG. 5). To this end, a-cyclodextrin can be partially methylated in that 1, 2, 3, 4 or 5 secondary hydroxyl groups are methylated. The methylated can also occur at the primary hydroxyl groups.

In some embodiments, the cyclodextrin is about 5% derivatised. In other embodiments, the derivatization is about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In some embodiments, the cyclodextrin is derivatised 1 to 24 times. In other embodiments, the cyclodextrin is derivatised 1 to 22 times, 1 to 20 times, 1 to 18 times, 1 to 16 times, 1 to 14 times, 1 to 12 times, 1 to 10 times, 1 to 9 times, 1 to 8 times, 1 to 7 times, 1 to 6 times, 1 to 5 times, 1 to 4 times, 1 to 3 times, or 1 to 2 times.

As used herein, cyclodextrins excludes cyclodextrin conjugates. A conjugate refers to a compound formed by the joining of two or more chemical compounds. Accordingly, a cyclodextrin conjugate refers to a compound formed by the joining of a cyclodextrin with another compound. The another compound can, for example, be an active agent, a fluorophore or a polymer.

In some embodiments, the cyclodextrin is methyl-β-cyclodextrin. In other embodiments, the cyclodextrin is 2-hydroxylpropyl-β-cyclodextrin. In other embodiments, the cyclodextrin is 3-hydroxylpropyl-β-cyclodextrin. In other embodiments, the cyclodextrin is dimethyl-β-cyclodextrin. In other embodiments, the cyclodextrin is trimethyl-β-cyclodextrin. In other embodiments, the cyclodextrin is sulfobutylether-β-cyclodextrin. In other embodiments, the cyclodextrin is carboxymethyl-β-cyclodextrin. In other embodiments, the cyclodextrin is a β-cyclodextrin selected from methyl-β-cyclodextrin, 2-hydroxylpropyl-β-cyclodextrin, or a combination thereof.

In some embodiments, the composition comprises cyclodextrin. In other embodiments, the composition consists essentially of cyclodextrin. In this regard, cyclodextrin is the sole active agent in the composition. In some embodiments, cyclodextrin is administered as the only active ingredient in the composition for treating lymphedema. Accordingly, in some embodiments, cyclodextrin is not used as an excipient in the composition. This, however, does not excludes the presence of excipients that do not play an active drug role in the composition.

In some embodiments, the cyclodextrin in the composition is provided in an uncomplexed or unoccupied state; i.e. an interior cavity of the cyclodextrin is unoccupied. As used herein, “complex” refers to the containment of a guest molecule within the cavity or space of the frustum shaped cyclodextrin. By doing so, the cyclodextrin is able to more easily exert its effect of complexing with lipids. The inventors have found that a composition as disclosed herein is particularly advantageous. It was also found that certain excipients are particularly advantageous in maintaining the cyclodextrin in its uncomplexed state until use.

Accordingly, the present invention provides a composition, the composition comprising cyclodextrin, wherein an interior cavity of the cyclodextrin is unoccupied.

In some embodiments, the composition consist essentially of cyclodextrin, wherein an interior cavity of the cyclodextrin is unoccupied.

In some embodiments, the interior cavity of the cyclodextrin has a diameter of about 5.5 Å to about 9.7 Å. In other embodiments, the diameter is about 5.7 Å to about 9.7 Å, about 5.7 Å to about 9.5 Å, about 5.7 Å to about 9.3 Å, about 5.7 Å to about 9.0 Å, about 5.7 Å to about 8.8 Å, about 5.7 Å to about 8.6 Å, about 5.7 Å to about 8.4 Å, about 5.7 Å to about 8.2 Å, about 5.7 Å to about 8.0 Å, about 5.9 Å to about 8.0 Å, about 6.0 Å to about 8.0 Å, about 6.2 Å to about 8.0 Å, about 6.4 Å to about 8.0 Å, about 6.6 Å to about 8.0 Å, about 6.8 Å to about 8.0 Å, about 7.0 Å to about 8.0 Å, about 7.2 Å to about 8.0 Å, about 7.4 Å to about 8.0 Å, about 7.6 Å to about 8.0 Å, or about 7.6 Å to about 7.8 Å.

In some embodiments, the cyclodextrin in the composition is complexed with an inert molecule. The complexation energy or interaction energy of this cyclodextrin-molecule complex is preferably lower than the complexation energy of the final cyclodextrin-lipid complex. Accordingly, complexation of the lipid with cyclodextrin is still favoured. It was found that by having an inert molecule complexed with cyclodextrin, the stability of the composition can be improved. It should be noted that in such cases, the cyclodextrin-molecule complex is not used in a host-guest complex sense in that the cyclodextrin is not used as a delivery platform/agent for carrying an active drug. Accordingly, the use of cyclodextrin (and any forms thereof) as a delivery platform is excluded. The inert molecule can be, for example, excipients that can be found within the composition. The inert molecule can serve to “protect” the cavity of the cyclodextrin such that it can efficiently form a host-guest complex with lipids when required.

As used herein, “complexation energy” or “interaction energy” refers to the energy between molecules A and B (ΔE^{AB}). It can be determined as the difference between the energy of the dimer (E^{A,B}) and the sum of the monomer energies (E^{A} + E^{B}).

Accordingly, in some embodiments, the composition comprising cyclodextrin, wherein the interior cavity of the cyclodextrin is unoccupied or occupied with an inert molecule.

In some embodiments, the composition consist essentially of cyclodextrin, wherein an interior cavity of the cyclodextrin is unoccupied or occupied with an inert molecule.

The composition can also comprise excipients. Excipients or additives can be added to enhance the solubility of cyclodextrin, to promote stability or to process the composition. Such additives and excipients include stabilisers, solubilisers, emulsifiers, surfactants, water-soluble polymers, pH modifiers, fillers, binders, pigments, disintegrants, antioxidants, preservatives (parabens, benzoic acid and benzoates), emollients (polyol such as sorbitol; natural oils such as castor oil, jojoba oil; waxes, synthetic organic polymers, petrolatum), silicones, penetration enhancers, lubricants and perfume. Other additives may also help the penetration of cyclodextrin in the skin, or at least penetrate the epidermis layer of the skin. Exemplary of such components are microcrystalline cellulose; metallic salts of acids such as aluminium stearate, calcium stearate, magnesium stearate, sodium stearate, and zinc stearate; fatty acids, hydrocarbons and fatty alcohols such as stearic acid, palmitic acid, liquid paraffin, stearyl alcohol, and palmitol; fatty acid esters such as glyceryl (mono-and di-) stearates, triglycerides, glyceryl (palmitic stearic) ester, sorbitan monostearate, saccharose monostearate, saccharose monopalmitate, and sodium stearyl fumarate, alkyl sulfates such as sodium lauryl sulfate and magnesium lauryl sulfate; polymers such as polyethylene glycols, polyoxethylene glycols, and polytetrafluoroethylene, and inorganic materials such as talc and dicalcium phosphate, and sodium starch glycolate.

Other excipients can be selected from carbomer (gelling agent), purified water (solvent), potassium sorbate (preservative), propylene glycol (permeation enhancer). Additional examples of excipients in topical formulations can be found in Chang et al., The AAPS Journal, 15:41-52 (2013), which is hereby incorporated by reference in its entirety. In some embodiments, the excipients may be selected from acrylates copolymer, carbomer 940, docusate sodium, edetate disodium, glycerin, poloxamer 182, propylene glycol, purified water, silicon dioxide, sodium hydroxide. In another variation, excipients may comprise carbomer, disodium EDTA, hydroxypropyl methylcellulose, laureth-4, sodium hydroxide, water. Additional examples of topical formulations can be found in Raphael et al., Therapeutic Delivery, Feb 6, 2:197-216 (2015), which is hereby incorporated by reference in its entirety.

In some embodiments, the weight ratio of cyclodextrin to composition is about 0.1%w/w to about 50%w/w. In other embodiments, the weight ratio is about 0.1%w/w to about 45%w/w, 0.5%w/w to about 45%w/w, 1%w/w to about 45%w/w, 1%w/w to about 40%w/w, 1%w/w to about 35%w/w, 1%w/w to about 30%w/w, 1%w/w to about 25%w/w, or 1%w/w to about 20%w/w. The weight ratio of the cyclodextrin to composition can for example be dependent on the type of formulation that the cyclodextrin is in.

Cyclodextrin can be administered to a subject as a pharmaceutically acceptable salt thereof. Suitable pharmaceutically acceptable salts include, but are not limited to salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium. In particular, the present invention includes within its scope cationic salts eg sodium or potassium salts, or alkyl esters (eg methyl, ethyl) of the phosphate group.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

In some embodiments, the composition comprises a saline solution. In other embodiments, the saline solution is a 0.9% saline solution. The salt can be NaCl or KCl. Other saline solutions can also be used, such as Ringer’s lactate solution, Acetated Ringer’s solution, Intravenous sugar solutions, 5% dextrose in normal saline (D5NS), 10% dextrose in normal saline (D10NS), 5% dextrose in half-normal saline (D5HNS), 10% dextrose in half-normal saline (D10HNS), Phosphate buffered saline (PBS), TRIS-buffered saline (TBS), Hank’s balanced salt solution (HBSS), Earle’s balanced salt solution (EBSS), Standard saline citrate (SSC), HEPES-buffered saline (HBS), and Gey’s balanced salt solution (GBSS).

In some embodiments, the weight ratio of the aqueous medium to the composition is about 0.1%w/w to about 50%w/w. In other embodiments, the weight ratio is about 0.1%w/w to about 45%w/w, 0.5%w/w to about 45 %w/w, 1%w/w to about 45%w/w, 1%w/w to about 40%w/w, 1%w/w to about 35%w/w, 1%w/w to about 30%w/w, 1%w/w to about 25%w/w, or 1 %w/w to about 20%w/w.

It will be appreciated that any compound that is a prodrug of cyclodextrin is also within the scope and spirit of the invention. Thus cyclodextrin can be administered to a subject in the form of a pharmaceutically acceptable pro-drug. The term “pro-drug” is used in its broadest sense and encompasses those derivatives that are converted in vivo to the compound of the invention. Such derivatives would readily occur to those skilled in the art. Other texts which generally describe prodrugs (and the preparation thereof) include: Design of Prodrugs, 1985, H. Bundgaard (Elsevier); The Practice of Medicinal Chemistry, 1996, Camille G. Wermuth et al., Chapter 31 (Academic Press); and A Textbook of Drug Design and Development, 1991, Bundgaard et al., Chapter 5, (Harwood Academic Publishers). For example, the hydroxyl groups on the cyclodextrin can be derivatised with a protecting group to form a methoxymethyl ether, tetrahydropranyl ether, t-butyl ether, allyl ether, benzyl ether, t-butyldimethylsilyl ether, t-butyldiphenylsilyl ether, acetic acid ester, pivalic acid ester, benzoic acid ester, acetonide or benzylidene acetal.

Cyclodextrin may be in crystalline form either as the free compound or as a solvate (e.g. hydrate) and it is intended that both forms are within the scope of the present invention. Methods of solvation are generally known within the art.

As mentioned, the composition is suitable for treating lymphedema. In other embodiments, the composition is suitable as a lymphedema prophylactic.

The composition is administered to the patient in a therapeutically effective amount. As used herein, a therapeutically effective amount is intended to include at least partially attaining the desired effect, or delaying the onset of, or inhibiting the progression of, or halting or reversing altogether the onset or progression of lymphedema.

As used herein, the term “effective amount” relates to an amount of cyclodextrin which, when administered according to a desired dosing regimen, provides the desired therapeutic activity. Dosing may occur at intervals of minutes, hours, days, weeks, months or years or continuously over any one of these periods. Suitable dosages may lie within the range of about 0.1 mg per kg of body weight to 100 g per kg of body weight per dosage, such as is in the range of 1 mg to 50 g per kg of body weight per dosage. In one embodiment, the dosage may be in the range of 1 mg to 20 g per kg of body weight per dosage. In another embodiment, the dosage may be in the range of 1 mg to 10 g per kg of body weight per dosage. In yet another embodiment, the dosage may be in the range of 1 mg to 50 g per kg of body weight per dosage, such as up to 5 g per body weight per dosage.

In some embodiments, the dosage is in the range of 0.5 g to 10 g per kg of body weight, 0.5 g to 9 g per kg of body weight, 0.5 g to 8 g per kg of body weight, 0.5 g to 7 g per kg of body weight, 0.5 g to 6 g per kg of body weight, 0.5 g to 5 g per kg of body weight, 0.5 g to 4 g per kg of body weight, or 0.5 g to 3 g per kg of body weight. In some embodiments, the composition is to be administered at a dose of about 0.5 g/kg to about 10 g/kg of body weight.

In some embodiments, the composition is to be administered on alternate days. In some embodiments, the composition is provided to a subject in need thereof as 2 to 10 doses every alternate days. In other embodiments, the composition is provided as 2, 4, 5, 6, 7, 8, 9 or 10 doses every alternate days.

In some embodiments, the composition is to be administered in at least 2 doses over at least 4 days. For example, the composition can be administered on the 1^{st} and 3^{rd} day of the treatment, at a single dose on the treatment day.

Preferred unit dosage composition or combinations are those containing a daily dose or unit, daily sub-dose, as herein above described, or an appropriate fraction thereof, of the active ingredient.

Other suitable dosage amounts and dosing regimens can be determined by the attending physician and may depend on the severity of the condition as well as the general age, health and weight of the patient to be treated.

The composition of the invention may be administered in a single dose or a series of doses. It is preferable to present the composition as a pharmaceutical composition. The formulation of such compositions is well known to those skilled in the art. The composition may contain any suitable carriers, diluents or excipients. These include all conventional solvents, dispersion media, fillers, solid carriers, coatings, antifungal and antibacterial agents, dermal penetration agents, surfactants, isotonic and absorption agents and the like.

The carrier must be pharmaceutically “acceptable” in the sense of being compatible with the other ingredients of the composition and not injurious to the patient. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

The composition can be suitable for parental administration. The parenteral route is any route that is not enteral (i.e. not administered via the gastrointestinal tract). For example, the composition can be suitable for subcutaneous delivery (application under the skin). A subcutaneous injection is administered as a bolus into the subcutis, the layer of skin directly below the dermis and epidermis, collectively referred to as the cutis. Alternatively, the composition can be suitable for epicutaneous delivery (application onto the skin), or intradermal delivery (application into the skin).

In other embodiments, the composition can be suitable for transdermal delivery. Transdermal is a route of administration in which an active ingredient is delivered across the skin for systemic distribution.

In some embodiments, the composition is applied to a transdermal patch. Transdermal is a route of administration in which the cyclodextrin is delivered across the skin. The cyclodextrin can be administered in the form of a patch. The patch can be an adhesive patch that is placed on the skin to deliver a specific dose of medication through the skin and optionally into the bloodstream. An advantage of a transdermal drug delivery route over other types of medication delivery such as oral, topical, intravenous, intramuscular is that the patch can provide a controlled release of the medication into the patient, usually through either a porous membrane covering a reservoir of medication or through body heat melting thin layers of medication embedded in the adhesive. Further, as the patch can be applied directly to the surface of the skin where lymphedema is observed, the treatment can be visually tracked.

In some embodiments, the composition is applied as a layer within the transdermal patch. In other embodiments, the composition is applied on a surface of the transdermal patch.

For example, the transdermal patch can comprise a backing layer, such as aluminum foil, polyethylene glycol terephthalate, polyethylene or non-woven fabric. The composition can be applied as a layer onto the backing layer. An adhesive layer can also be present for contacting to a skin of a patient. The composition layer and the adhesive layer can be in any form and configuration as long as the composition can be delivered to the patient via the skin. Alternatively, the composition and the adhesive can be inseparably mixed to form a single layer. The adhesive can be an acrylic based adhesive or a silicon based adhesive.

In some embodiments, the weight ratio of cyclodextrin in the transdermal patch is about 0.1%w/w to about 50%w/w. In other embodiments, the weight ratio is about 0.1%w/w to about 45%w/w, 0.5%w/w to about 45%w/w, 1%w/w to about 45%w/w, 1%w/w to about 40%w/w, 1%w/w to about 35%w/w, 1 %w/w to about 30%w/w, 1%w/w to about 25%w/w, or 1%w/w to about 20 %w/w.

The composition of the invention can be administered to the subject using any known administration technique. For example, the composition can be administered as a continuous delivery (allows carefully regulated amount of fluid over a prolonged period), intermittent delivery (a dosage given for shorter periods at set intervals) or as a direct delivery (to deliver single dose or bolus). For example, the composition may be administered at a dose of up to 20 g/m².

Injectables for such use can be prepared in conventional forms, either as a liquid solution or suspension or in a solid form suitable for preparation as a solution or suspension in a liquid prior to injection, or as an emulsion. Carriers can include, for example, water, saline (e.g., normal saline (NS), phosphate-buffered saline (PBS), balanced saline solution (BSS)), sodium lactate Ringer’s solution, dextrose, glycerol, ethanol, and the like; and if desired, minor amounts of auxiliary substances, such as wetting or emulsifying agents, buffers, and the like can be added. Proper fluidity can be maintained, for example, by using a coating such as lecithin, by maintaining the required particle size in the case of dispersion and by using surfactants.

In some embodiments, the composition is an intradermal composition. The intradermal composition is suitable for intradermal injection into a patient in need thereof. Intradermal injection is a shallow or superficial injection of a substance into the dermis, which is located between the epidermis and the hypodermis. It was found that the intradermal route is associated with fast uptake of cyclodextrin systemically compared to subcutaneous injections, and hence the response time for treating lymphedema is faster. Additionally, the body’s reaction to the composition is more easily visible since it is closer to the surface.

In some embodiments, the intradermal composition comprises an aqueous medium. The term ‘aqueous medium’ used herein refers to a water based solvent or solvent system, and which comprises of mainly water. Such solvents can be either polar or non-polar, and/or either protic or aprotic. Solvent systems refer to combinations of solvents which resulting in a final single phase. Both ‘solvents’ and ‘solvent systems’ can include, and is not limited to, pentane, cyclopentane, hexane, cyclohexane, benzene, toluene, dioxane, chloroform, diethylether, dichloromethane, tetrahydrofuran, ethyl acetate, acetone, dimethylformamide, acetonitrile, dimethyl sulfoxide, nitromethane, propylene carbonate, formic acid, butanol, isopropanol, propanol, ethanol, methanol, acetic acid, ethylene glycol, diethylene glycol or water. Water based solvent or solvent systems can also include dissolved ions, salts and molecules such as amino acids, proteins, sugars and phospholipids. Such salts may be, but not limited to, sodium chloride, potassium chloride, ammonium acetate, magnesium acetate, magnesium chloride, magnesium sulfate, potassium acetate, potassium chloride, sodium acetate, sodium citrate, zinc chloride, HEPES sodium, calcium chloride, ferric nitrate, sodium bicarbonate, potassium phosphate and sodium phosphate. As such, biological fluids, physiological solutions and culture medium also falls within this definition.

In some embodiments, the weight ratio of cyclodextrin to intradermal composition is about 0.1%w/w to about 50%w/w. In other embodiments, the weight ratio is about 0.1%w/w to about 45%w/w, 0.5%w/w to about 45 %w/w, 1%w/w to about 45%w/w, 1%w/w to about 40%w/w, 1%w/w to about 35%w/w, 1%w/w to about 30%w/w, 1%w/w to about 25%w/w, or 1 %w/w to about 20%w/w.

In some embodiments, the intradermal composition comprises excipients. The excipients can help improve the solubility and/or stability of cyclodextrin in the composition. For example, excipients such as carboxymethylcellulose sodium, microcrystalline cellulose, cresol, methylparaben, and/or propylparaben can be used.

In some embodiments, the weight ratio of excipients to intradermal composition is about 0.1%w/w to about 50%w/w. In other embodiments, the weight ratio is about 0.1%w/w to about 45%w/w, 0.5%w/w to about 45%w/w, 1%w/w to about 45%w/w, 1%w/w to about 40%w/w, 1%w/w to about 35%w/w, 1 %w/w to about 30%w/w, 1%w/w to about 25%w/w, or 1%w/w to about 20 %w/w.

The composition of the invention may be suitable for parenteral administration include aqueous and non-aqueous isotonic sterile injection solutions which may contain anti-oxidants, buffers, bactericides and solutes which render the compound, composition or combination isotonic with the blood of the intended recipient; and aqueous and non-aqueous sterile suspensions which may include suspending agents and thickening agents. The compound, composition or combination may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

A person skilled in the art will appreciate that other means for injecting and/or administering the composition can also be used. These other means can include, for example, medical delivery devices. These devices and methods can include, for example, biodegradable polymer delivery members that are inserted into a body of the subject for long term delivery of medicaments.

Other modes of administration including topical administration may also be possible. A topical delivery refers to an application to a particular place on or in the body. In most applications, topical administration means application to body surfaces such as the skin or mucous membranes to treat ailments via a large range of classes including creams, foams, gels, lotions, and ointments. For example, solutions or suspensions of the composition of the invention may be formulated as a membranous patch, which is applied directly to a skin surface of the subject. Alternatively, the composition can be a cream or gel which is applied to a skin surface of the subject. Topical application typically involves administering cyclodextrin in an amount between 0.1 mg and 100 g.

It is believed that subcutaneous injection likely reflects topical application since the same layers of the skin such as dermis are targeted by these two routes of administration.

In some embodiments, the composition is a topical composition. A topical medication is a medication that can be applied to a particular place on or in the body. Most often topical administration means application to body surfaces such as the skin or mucous membranes to treat ailments via a large range of classes including creams, foams, gels, lotions, and ointments. Many topical medications are epicutaneous, meaning that they are applied directly to the skin. Topical medications may also be inhalational, such as asthma medications, or applied to the surface of tissues other than the skin, such as eye drops applied to the conjunctiva, or ear drops placed in the ear, or medications applied to the surface of a tooth.

In some embodiments, the weight ratio of cyclodextrin to topical composition is about 0.1%w/w to about 50%w/w. In other embodiments, the weight ratio is about 0.1%w/w to about 45%w/w, 0.5%w/w to about 45%w/w, 1%w/w to about 45%w/w, 1%w/w to about 40%w/w, 1%w/w to about 35%w/w, 1 %w/w to about 30%w/w, 1%w/w to about 25%w/w, or 1%w/w to about 20 %w/w.

In some embodiments, the topical composition comprises excipients. The excipients can help improve the solubility and/or stability of cyclodextrin in the composition. For example, the excipients can be an emulsifier and/or a thickening agent. For example, the excipients can be Arabic gum, acetic acid, acetone, acetyltributyl citrate, agar, ethanol, alginic acid, almond oil, alpha tocopherol, aluminium monostearate, aluminium stearate, aluminium oxide, ascorbic acid, ascorbyl palmitate, bentonite, benzalkonium chloride, benzethonium chloride, benzoic acid, benzyl alcohol, benzyl benzoate, boric acid, bronopol, butylated hydroxyanisole, butylated hydroxytoluene, butylene glycol, butylparaben, calcium acetate, calcium alginate, calcium chloride, calcium hydroxide, calcium lactate, carbomer, carboxymethylcellulose sodium, carrageenan, castor oil, microcrystalline cellulose, ceresin, cetostearyl alcohol, cetrimide, cetyl alcohol, chlorocresol, chloroxylenol, chlorosterol, citric acid, colloidal silicon dioxide, cresol, crospovidone, cyclomethicone, denatonium benzoate, dibutyl phthalate, diethanolamine, dimethicone, dimethyl phthalate, dimethylacetamide, disodium edetate, docusate sodium, ethyl acetate, ethyl lactate, ethyl oleate, ethylene vinyl acetate, ethylparaben, gelatin, glycerine, glyceryl monooleate, glyceryl monostearate, glycofurol, hydroxyethyl cellulose, hydroxypropyl cellulose, hypromellose, imidurea, isopropyl alcohol, isopropyl myristate, isopropyl palmitate, kaolin, lactic acid, lanolin, lecithin, linoleic acid, silicate, malic acid, mannitol, methylcellulose, methylparaben, monothioglycerol, myristic acid, myristyl alcohol, oleic acid, oleyl alcohol, palmitic acid, paraffin, petrolatum, phenoxyethanol, poloxamer, polycarbophil, polyethylene glycol, polymethylacrylate, polyoxyglyceride, polyvinyl alcohol, povidone, propylene glycol, propylparaben, pyrrolidone, sodium hydroluronate, sodium lactate, sodium lauryl sulfate, sorbitan ester, sorbitol, starch, tricaprylin, triethylanolamine, xanthan gum, or xylitol.

In some embodiments, the weight ratio of excipients to topical composition is about 0.1%w/w to about 50%w/w. In other embodiments, the weight ratio is about 0.1%w/w to about 45%w/w, 0.5%w/w to about 45%w/w, 1%w/w to about 45%w/w, 1%w/w to about 40%w/w, 1%w/w to about 35%w/w, 1 %w/w to about 30%w/w, 1%w/w to about 25%w/w, or 1%w/w to about 20 %w/w.

The composition of the invention may be suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal patches may also be used to administer the compounds of the invention.

The present invention provides a method of treating lymphedema comprises administering an effective amount of a composition to a patient in need thereof, the composition comprising cyclodextrin or a pharmaceutically acceptable salt, solvate or prodrug thereof;

wherein an interior cavity of the cyclodextrin is unoccupied.

In some embodiments, the method of treating lymphedema comprising administering an effective amount of a composition to a patient in need thereof, the composition consisting essentially of cyclodextrin or a pharmaceutically acceptable salt, solvate or prodrug thereof.

In some embodiments, the method of treating lymphedema comprising administering an effective amount of a composition to a patient in need thereof, the composition consisting essentially of cyclodextrin or a pharmaceutically acceptable salt, solvate or prodrug thereof, wherein an interior cavity of the cyclodextrin is unoccupied.

The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

The term “administering” refers to contacting, applying, injecting, transfusing or providing an inhibitor as referred to herein to a subject.

The present invention also provides a composition for treating lymphedema in a patient in need thereof is provided, the composition comprising cyclodextrin or a pharmaceutically acceptable salt, solvate or prodrug thereof;

wherein an interior cavity of the cyclodextrin is unoccupied.

In some embodiments, the composition is for treating lymphedema in a patient in need thereof, the composition consisting essentially of cyclodextrin or a pharmaceutically acceptable salt, solvate or prodrug thereof.

In some embodiments, the composition is for treating lymphedema in a patient in need thereof, the composition consisting essentially of cyclodextrin or a pharmaceutically acceptable salt, solvate or prodrug thereof, wherein an interior cavity of the cyclodextrin is unoccupied.

The present invention also provides a use of a composition in the manufacture of a medicament for treating lymphedema is provided, the composition comprising cyclodextrin or a pharmaceutically acceptable salt, solvate or prodrug thereof;

wherein an interior cavity of the cyclodextrin is unoccupied.

In some embodiments, the use of a composition is in the manufacture of a medicament for treating lymphedema, the composition consisting essentially of cyclodextrin or a pharmaceutically acceptable salt, solvate or prodrug thereof.

In some embodiments, the use of a composition is in the manufacture of a medicament for treating lymphedema, the composition consisting essentially of cyclodextrin or a pharmaceutically acceptable salt, solvate or prodrug thereof, wherein an interior cavity of the cyclodextrin is unoccupied.

In some embodiments, the lymphedema is primary lymphedema. In other embodiments, the lymphedema is secondary lymphedema. In other embodiments, the lymphedema is selected from primary lymphedema or secondary lymphedema.

In some embodiments, the composition to be administered ameliorates or reduces tissue edema, lymphatic function, lipid accumulation, tissue cholesterol, fibrosis, inflammation, or a combination thereof.

A method of classifying primary and secondary lymphedema is as follows:

TABLE 1

Staging of lymphoedema from International Society of Lymphology (2013)

| | | | |
|---|---|---|---|
| | Presentation | Severity | Management |
| Stage 0 (latent sub-clinical stage) | No overt swelling but lymphatic pathways have been disrupted | | |
| Stage 1 (early stage) | Mild pitting oedema that resolves with elevation | Mild-<20% increase in excess limb volume | Compression hosiery, exercise, simple-or self-lymphatic drainage, preventative skin care |
| Stage 2 | Swelling does not resolve with elevation. Less evidence of pitting as fibrosis development occurs | Moderate -20-40% increase in excess limb volume | Custom-made hosiery/ compression bandaging, exercise, self-/manual lymphatic drainage, skin care |
| Stage 3 (late stage) | Non-pitting with skin changes (papillomata, fibrosis, hyperkeratosis). | Severe -> 40% increase in excess limb volume | Compression bandaging, skin care, exercise, manual lymphatic drainage |

The staging system helps to identify the severity of lymphedema. With the assistance of medical imaging apparatus, such as MRI or CT, staging can be established by the physician, and therapeutic or medical interventions may be applied.

Both lymphoedema and chronic oedema can have a devastating physical and psychosocial effect on both the patients and their families. The extra weight of the swollen limb(s) can affect gait, mobility, and cause pain in surrounding joints and muscles. There is often difficulty with clothing and footwear, and this causes body image and social issues. Many find it difficult to maintain their current employment or find alternatives and this can lead to, or exacerbate, sedentary lifestyles. Other complications include malignant transformation and recurrent skin infections.

In some embodiments, the composition is to be administered to a patient in need thereof who has lymphedema with a severity of stage 2 or less. In other embodiments, the composition is to be administered to a patient in need thereof who has lymphedema with a severity of stage 1 or less.

### EXAMPLES

### Methodology

To evaluate the effect of cyclodextrin on secondary lymphedema, an animal model of lymphatic insufficiency that mimics human pathophysiology of lymphedema was used, e.g adipose tissue remodeling, lipid accumulation, inflammation and fibrosis. This mouse model can be apoE lipoprotein E deficient mice. It is shown that the mice exhibit impaired skin lymphatic transport, tissue swelling, lipid accumulation and inflammation in the tissue. It is believed that this lymphatic insufficiency leads to adipose tissue expansion and then loss and fibrosis as also observed in human secondary lymphedema. To confirm this, female apoE-/- mice were treated with 2 g/kg body of cyclodextrin (saline) subcutaneously or vehicle alone (saline) subcutaneously every alternative day for a total of 2, 4 or 8 doses. Mice were euthanized, tissue swelling was observed macroscopically and lymphatic transport was examined by following up the transport of FITC dextran from the front footpad to the draining lymph nodes. Skin tissues was also examined histologically for lipid content (Oil red O staining).

### Results and Analysis

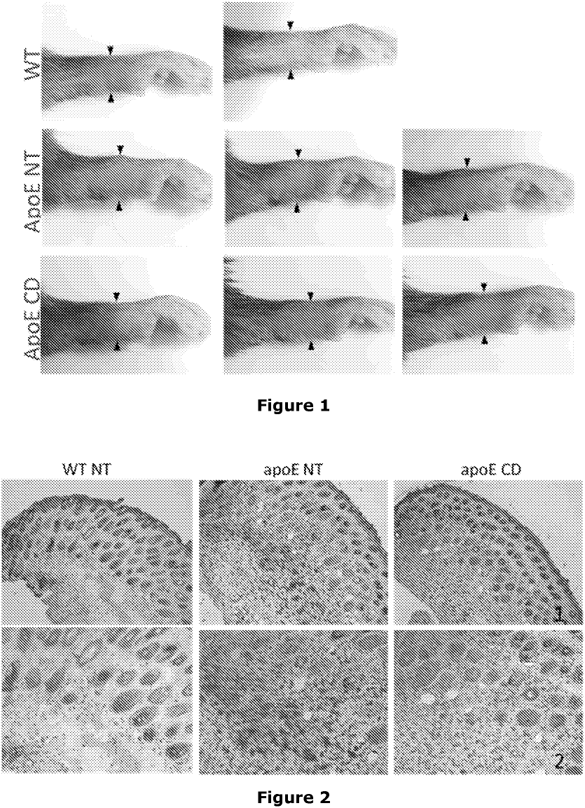

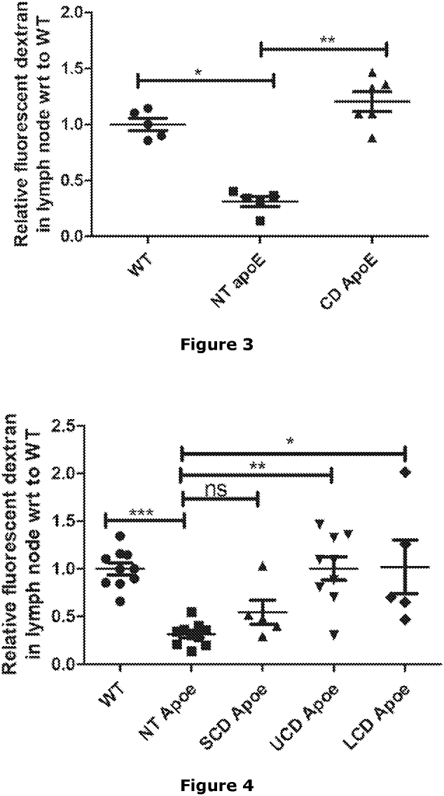

The effect of 4 doses of cyclodextrin on tissue edema, lipid accumulation and lymphatic transport in apoE-/- mice was tested (FIGS. 1, 2 and 3). It is shown that 4 doses improved significantly tissue edema in the footpad (FIG. 1) and decreased lipid accumulation by the reduction in Oild red O staining in tissue from mice treated with cyclodextrin (FIG. 2). Moreover, it is demonstrated that cyclodextrin completely restored lymphatic transport in apoE-/- mice (FIG. 3), and thus improve tissue swelling conditions.

FIG. 1 illustrates images of footpads from female wild-type (WT) or apoE-/-mice after receiving subcutaneous injection of cyclodextrin (CD; 2 g/kg body weight) or saline (control, NT) every alternative day for a total of 4 doses and harvested when mice were 17 weeks old on high fat diet. Swelling of the footpad of the mice was observed macroscopically (arrow heads: swelling region). ApoE-/- mice treated with cyclodextrin (apoE CD) showed decreased footpad swelling compared to non-treated apoE-/- mice (apoE NT).

FIG. 2 illustrates microscopic images of female WT or apoE-/- mice after receiving subcutaneous injection of cyclodextrin (CD; 2 g/kg body weight) or saline (control, NT) every alternative day for a total of 4 doses and harvested when mice were 17 weeks old on high fat diet. Section of footpad skin were stained for neutral lipid with Oil red O (red staining) to identify lipid accumulation in the tissue. Non treated ApoE-/- mice (apoE NT) exhibited accumulation of lipids in skin compared to non treated wild-type mice (WT NT). In contrast, apoE-/- mice treated with cyclodextrin (apoE CD) showed decreased accumulation of lipids compared to non treated apoE-/- mice.

FIG. 3 plots the relative fluorescence in the lymph nodes of respective female WT or apoE-/- mice after receiving subcutaneous injection of cyclodextrin (CD; 2 g/kg body weight) or Saline (control, NT) every alternative day for a total of 4 doses. The mice were harvested when the mice were 17 weeks old on high fat diet. Lymphatic drainage was assessed by measuring the transport of fluorescent dextran from the footpad (site of injection) to the draining lymph node.

Next, the ameliorating effect of cyclodextrin on lymphatic function was tested which was observed when apoE-/- mice received treatment on alternate day with 2 doses, 4 doses or 8 doses. Two doses improved lymphatic function but in lesser extent compared to 4 doses whereas 8 doses did not further enhance lymphatic function in apoE-/- mice compared to 4 doses which was already restoring completely lymphatic function in these mice (FIG. 4)

FIG. 4 plots the relative fluorescence in the lymph nodes of the respective mice under a different dosage regime. Female wild-type (WT) or apoE-/- mice at 17 weeks old received subcutaneously 2, 4 or 8 doses of cyclodextrin (2 g/kg body weight) every alternate days (SCD: 2 doses; UCD: 4 doses (usual treatment) and LCD: 8 doses. Non treated (NT) treated WT and apoE mice received saline for 4 doses. Lymphatic transport was assessed as stated in FIG. 3. Two doses of cyclodextrin was sufficient to improve lymphatic function in apoE-/- mice but 4 doses completely restored it. Prolonged treatment did not improve further lymphatic function in apoE-/- mice.

It is thus demonstrated that subcutaneous treatment with cyclodextrin formulations can reduce lipid accumulation in lymphedema skin and tissue edema. Moreover, it restores lymphatic transport. Since lipid accumulation initiates adipose tissue remodeling and the subsequent inflammation and fibrosis in human lymphedema, it is believed that cyclodextrin compositions can be used for treating lymphedema.

The treatment of apoE-/- mice, an animal model that recapitulates lymphedema pathophysiology, with cyclodextrin reduces tissue cholesterol but does not affect plasma cholesterol levels showing that cyclodextrin is potent to clear cholesterol from the skin (FIG. 6).

FIG. 6 shows the total cholesterol concentration analysed in the front footpad of wild-type (n=4), apoE-/- mice (n=4), 4-dose treated apoE-/- mice (n=2) and 8-dose treated apoE-/- mice (n=5) at 17 weeks old. *p<0.05. There was decreased total cholesterol levels in the front foot pad of apoE-/- mice as the dosage of cyclodextrin increases. Cyclodextrin was administrated subcutaneously alternate days.

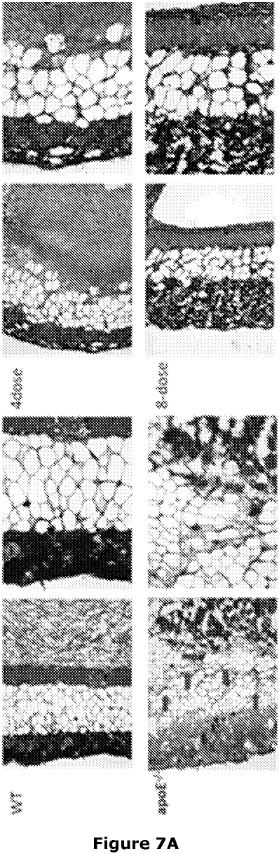

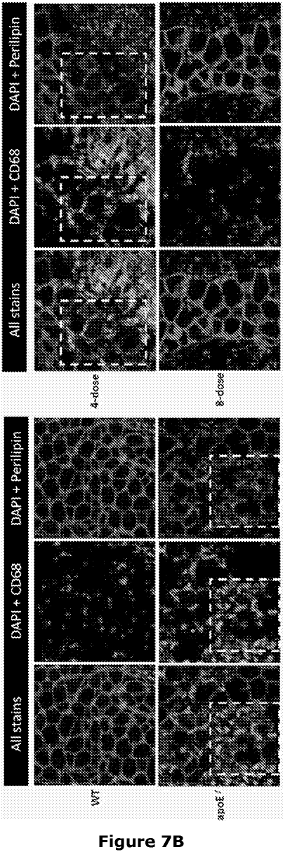

The formulation was assessed if it can reverses some tissue changes associated with lymphedema including fibrosis and inflammation. Treatment of apoE-/-mice with cyclodextrin, particularly the 8 doses regime, reduces fibrosis (FIG. 7A) and inflammation (FIG. 7B) as noted by the reduced collagen deposition and infiltration of macrophages in the adipose tissue. These data shows that lymphedema can be improved by cyclodextrin treatment.

FIG. 7A shows Masson’s Trichrome staining of the back skin of 17 week-old wild-type, non-treated apoE-/- mice, 4 - dose treated mice and 8-dose treated mice. Collagen was stained in blue, muscle and cytoplasm was stained in red and the nucleus was stained in black.

FIG. 7B shows immunofluorescence staining of the back skin of 17 week-old wild-type, non-treated apoE-/- and cyclodextrin-treated apoE-/- mice. CD68 is a pan-macrophage marker, perilipin was used to identify adipocytes and DAPI stains for the nucleus. As cyclodextrin dosage increases, as shown by CD68 staining, the extent of macrophage accumulation and crown-like structures (white boxes) decreases.

The efficiency of treatment with cyclodextrin with 8 doses was tested with lymphatic dysfunction and disease at a greater severity. Mice at 42 weeks of age instead of 17 weeks of age were tested. At 8 doses, it was observed that cyclodextrin was not able to improve lymphatic function and ameliorate tissue changes such as tissue swelling.

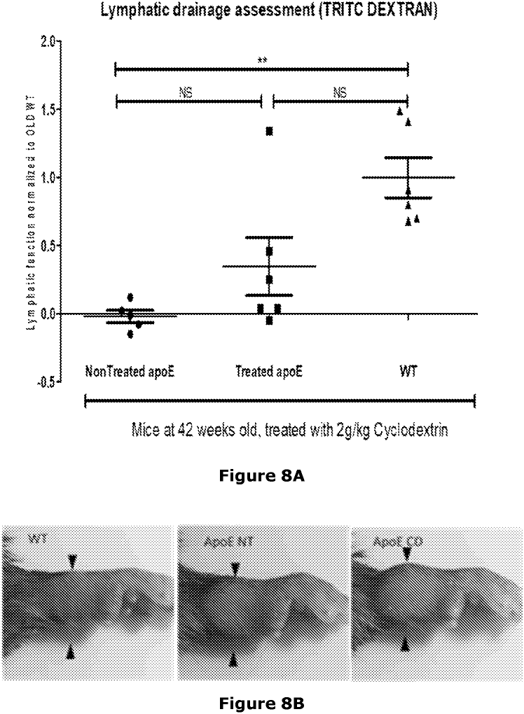

FIG. 8A shows lymphatic function assessed in 42 weeks old WT, nontreated apoE-/- and cyclodextrin treated apoE-/- for 8 doses by quantifying the amount of FITC dextran transported from the footpad to the draining lymph node.

FIG. 8B shows footpad tissue swelling was examined macroscopically in 42 weeks old WT, nontreated apoE-/- and cyclodextrin treated apoE-/- for 8 doses.

Altogether, these data further support the potential therapeutic benefit of cyclodextrin for lymphedema by decreasing tissue cholesterol and subsequently adipose tissue remodelling, fibrosis and inflammation.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A method of treating lymphedema comprising administering an effective amount of a composition to a patient in need thereof, the composition comprising cyclodextrin or a pharmaceutically acceptable salt, solvate or prodrug thereof;
wherein an interior cavity of the cyclodextrin is unoccupied.

2. (canceled)

3. (canceled)

4. The method according to claim 1, wherein the composition to be administered ameliorates or reduces tissue edema, lymphatic function, lipid accumulation, tissue cholesterol, fibrosis, inflammation, or a combination thereof.

5. The method according to claim 1, wherein the composition is to be administered on alternate days; or wherein the composition is to be administered in at least 2 doses over at least 4 days.

6. (canceled)

7. The method according to claim 1, wherein the composition is to be administered at a dose of about 0.5 g/kg to about 10 g/kg.

8. The method according to claim 1, wherein the composition is to be administered to a patient in need thereof who has lymphedema with a severity of stage 2 or less.

9. A composition comprising cyclodextrin;
wherein an interior cavity of the cyclodextrin is unoccupied;
wherein the composition is an intradermal composition, topical cream or topical gel;
wherein a weight ratio of cyclodextrin to composition is about 0.1%w/w to about 50 %w/w; and
wherein when the composition is a topical cream or topical gel, the composition further comprises a penetration enhancer.

10. (canceled)

11. The composition according to claim 9, wherein the cyclodextrin is derivatised 1 to 24 times with alkyl, alkenyl, alkynyl, alkoxy, hydroalkyl, halo, haloalkyl, haloalkenyl, haloalkynyl, alkylacyl, alkenylacyl, alkynylacyl, aryl, or alkylaryl, or preferably.

12. The composition according to claim 11, wherein the cyclodextrin is derivatised 1 to 24 times with methyl, ethyl, propyl, hydromethyl, hydroethyl, hydropropyl, fluoro, chloro, bromo, benzyl, or phenyl.

13. (canceled)

14. The composition according to claim 9, wherein the cyclodextrin is a β-cyclodextrin selected from methyl-β-cyclodextrin, 2-hydroxylpropyl-β-cyclodextrin, or a combination thereof.

15. (canceled)

16. (canceled)

17. The composition according claim 9, further comprising an excipient selected from stabilisers, solubilisers, emulsifiers, surfactants, water-soluble polymers, pH modifiers, fillers, binders, pigments, disintegrants, antioxidants, preservatives, emollients, silicones, penetration enhancers, lubricants and perfume.

18. The composition according to claim 17, wherein the excipient is selected from microcrystalline cellulose; metallic salts of acids such as aluminium stearate, calcium stearate, magnesium stearate, sodium stearate, and zinc stearate; fatty acids, hydrocarbons and fatty alcohols such as stearic acid, palmitic acid, liquid paraffin, stearyl alcohol, and palmitol; fatty acid esters such as glyceryl (mono- and di-) stearates, triglycerides, glyceryl (palmitic stearic) ester, sorbitan monostearate, saccharose monostearate, saccharose monopalmitate, and sodium stearyl fumarate, alkyl sulfates such as sodium lauryl sulfate and magnesium lauryl sulfate; polymers such as polyethylene glycols, polyoxethylene glycols, and polytetrafluoroethylene, and inorganic materials such as talc and dicalcium phosphate, and sodium starch glycolate.

19. (canceled)

20. The composition according to claim 9, wherein the intradermal composition comprises an aqueous medium.

21. The composition according to claim 20, wherein the aqueous medium is selected from 0.9% NaCl saline solution 0.9% KCl saline solution, Ringer’s lactate solution, Acetated Ringer’s solution, Intravenous sugar solutions, 5% dextrose in normal saline (D5NS), 10% dextrose in normal saline (D10NS), 5% dextrose in half-normal saline (D5HNS), 10% dextrose in half-normal saline (D10HNS), Phosphate buffered saline (PBS), TRIS-buffered saline (TBS), Hank’s balanced salt solution (HBSS), Earle’s balanced salt solution (EBSS), Standard saline citrate (SSC), HEPES-buffered saline (HBS), and Gey’s balanced salt solution (GBSS).

22. The composition according to claim 20, wherein the weight ratio of the aqueous medium to the composition is about 0.1%w/w to about 50 %w/w.

23. The composition according to claim 9, wherein the intradermal composition comprises an excipient selected from carboxymethylcellulose sodium, microcrystalline cellulose, cresol, methylparaben, and propylparaben.

24. The composition according to claim 23, wherein the weight ratio of excipients to intradermal composition is about 0.1%w/w to about 50%w/w.

25. (canceled)

26. The composition according to claim 9, wherein the topical gel or topical cream further comprises an excipient selected from a carrier, an emulsifier and/or a thickening agent.

27. The composition according to claim 26, wherein the topical composition further comprises an excipient selected from Arabic gum, acetic acid, acetone, acetyltributyl citrate, agar, ethanol, alginic acid, almond oil, alpha tocopherol, aluminium monostearate, aluminium stearate, aluminium oxide, ascorbic acid, ascorbyl palmitate, bentonite, benzalkonium chloride, benzethonium chloride, benzoic acid, benzyl alcohol, benzyl benzoate, boric acid, bronopol, butylated hydroxyanisole, butylated hydroxytoluene, butylene glycol, butylparaben, calcium acetate, calcium alginate, calcium chloride, calcium hydroxide, calcium lactate, carbomer, carboxymethylcellulose sodium, carrageenan, castor oil, microcrystalline cellulose, ceresin, cetostearyl alcohol, cetrimide, cetyl alcohol, chlorocresol, chloroxylenol, chlorosterol, citric acid, colloidal silicon dioxide, cresol, crospovidone, cyclomethicone, denatonium benzoate, dibutyl phthalate, diethanolamine, dimethicone, dimethyl phthalate, dimethylacetamide, disodium edetate, docusate sodium, ethyl acetate, ethyl lactate, ethyl oleate, ethylene vinyl acetate, ethylparaben, gelatin, glycerine, glyceryl monooleate, glyceryl monostearate, glycofurol, hydroxyethyl cellulose, hydroxypropyl cellulose, hypromellose, imidurea, isopropyl alcohol, isopropyl myristate, isopropyl palmitate, kaolin, lactic acid, lanolin, lecithin, linoleic acid, silicate, malic acid, mannitol, methylcellulose, methylparaben, monothioglycerol, myristic acid, myristyl alcohol, oleic acid, oleyl alcohol, palmitic acid, paraffin, petrolatum, phenoxyethanol, poloxamer, polycarbophil, polyethylene glycol, polymethylacrylate, polyoxyglyceride, polyvinyl alcohol, povidone, propylene glycol, propylparaben, pyrrolidone, sodium hydroluronate, sodium lactate, sodium lauryl sulfate, sorbitan ester, sorbitol, starch, tricaprylin, triethylanolamine, xanthan gum, or xylitol.

28. The composition according to claim 27, wherein the weight ratio of excipients to topical gel or topical cream is about 0.1%w/w to about 50%w/w.

29. (canceled)

30. (canceled)

31. The method according to claim 1, wherein the composition is subcutaneously deliverable to the patient in need thereof.

32. The method according to claim 1, wherein the composition is an intradermal composition, topical cream or topical gel.
